(19) United States
(12) Patent Application Publication
(10) Pub. No.: US 2022/0370365 A1
(43) Pub. Date: Nov. 24, 2022

(54) SOLID PREPARATION CONTAINING TAFAMIDIS AND METHOD FOR PRODUCING THE SAME

(71) Applicant: SAWAI PHARMACEUTICAL Co., Ltd., Osaka City (JP)

(72) Inventors: Wataru IZUI, Osaka City (JP); Daiki BIRUKAWA, Osaka City (JP); Ayane NATSUME, Osaka City (JP)

(21) Appl. No.: 17/747,276

(22) Filed: May 18, 2022

(30) Foreign Application Priority Data
May 21, 2021 (JP) 2021-86377

(51) Int. Cl. A61K 9/20; A61K 31/423

(52) U.S. Cl. CPC A61K 9/2018; A61K 31/423; A61K 9/2009; A61K 9/2054; A61K 9/2027; A61K 9/2095

(57) ABSTRACT

A solid preparation containing tafamidis includes tafamidis, and a carrier containing one or more selected from a group consisting of magnesium aluminometasilicate, microcrystalline cellulose, low substituted hydroxypropyl cellulose, crospovidone and calcium silicate, and a method for producing the same are provided.

### CROSS-REFERENCE TO RELATED APPLICATIONS

This application claims the benefit of priority to Japanese Patent Application No. 2021-86377, filed on May 21, 2021, the entire contents of which are incorporated herein by reference.

### FIELD

The present invention relates to a solid preparation containing tafamidis and a method for producing the same.

### BACKGROUND

Tafamidis (2-(3,5-dichlorophenyl)-1,3-benzoxazole-6-carboxylic acid) binds to the thyroxin binding site of transthyretin (TTR) tetramers to form stable TTR tetramers. It is a transthyretin-type familial amyloid polyneuropathy (TTR-FAP) therapeutic agent that suppresses the formation of amyloid and its deposition in tissues by inhibiting dissociation into monomers. There is a soft capsule containing tafamidis, for example, Vyndaqel (registered trademark) capsules (Vyndaqel (registered trademark) capsule Interview Form, revised in August 2020 (8th edition)). The Vyndaqel capsule is a formulation in which tafamidis meglumine is dispersed in macrogol 400, polysorbate 80, and sorbitan monooleate and encapsulated in a soft capsule. The soft capsule contains 20.0 mg of tafamidis meglumine (12.2 mg of tafamidis).

Since the soft capsule preparation requires equipment for encapsulating the drug substance-containing solution or suspension, the manufacturing cost is high and the control of the encapsulation process is complicated. In addition, since the soft capsule preparation cannot be administered in divided doses, it is difficult to adjust the dose, and it is not always convenient. Therefore, it is desired to formulate the product in a form that eliminates these defects, such as tablets and granules.

### SUMMARY

One of the objects of the present invention is to provide a solid preparation containing tafamidis in which the decrease in the elution rate from the tafamidis preparation is suppressed and a method for producing the same.

According to one embodiment of the present invention, a solid preparation containing tafamidis is provided including tafamidis, and a carrier containing one or more selected from a group consisting of magnesium aluminometasilicate, microcrystalline cellulose, low substituted hydroxypropyl cellulose, crospovidone and calcium silicate. The solid preparation containing tafamidis may further includes polysorbate 80 and sorbitan monooleate.

According to one embodiment of the present invention, a method for producing a powdered preparation containing tafamidis is provided including dispersing tafamidis in a dispersion medium to obtain a tafamidis dispersion, and adsorbing the obtained tafamidis dispersion in a carrier containing one or more selected from a group consisting of magnesium aluminometasilicate, microcrystalline cellulose, low substituted hydroxypropyl cellulose, crospovidone and calcium silicate. The dispersion medium may include polysorbate 80 and sorbitan monooleate. The dispersion medium may include water.

The carrier may include one or more selected from a group consisting of magnesium aluminometasilicate, microcrystalline cellulose, low-substituted hydroxypropyl cellulose and crospovidone. The carrier may be magnesium aluminometasilicate.

According to one embodiment of the present invention, a method for producing a tablet containing tafamidis is provided including tableting a mixture containing a powder containing tafamidis.

### DESCRIPTION OF EMBODIMENTS

Hereinafter, a solid preparation containing tafamidis and a method for producing the same according to the present invention will be described in detail. However, the solid preparation containing tafamidis and the method for producing the same of the present invention are not to be construed as being limited to the description contents of the embodiments and examples shown below.

As shown in the Examples described later, the present inventors have investigated various carriers for adsorbing a tafamidis dispersion in which tafamidis is dispersed in a dispersion medium in order to produce the solid preparation containing tafamidis such as tablets and granules, and found that specific carriers such as magnesium aluminometasilicate are useful as adsorption carriers. In addition, by adsorbing the tafamidis dispersion in a specific carrier to produce a powdered preparation containing tafamidis, the decrease in the elution rate of tafamidis from the preparation is suppressed, and the powdered preparation that is optimal for tablet preparation can be easily produced.

Tafamidis of the present embodiment may be a free form of tafamidis, or tafamidis meglumine (2-(3,5-dichlorophenyl)-1,3-benzoxazole-6-carboxylic acid mono (1-deoxy-1-methylamino-D-glucitol)). However, the present embodiment is not limited thereto and tafamidis may be its pharmacologically acceptable salt or solvate thereof. The term tafamidis in the present application may include its free form and its pharmacologically acceptable salt or solvate. The content of tafamidis meglumine can be appropriately selected according to the expected therapeutic effect, and, one tablet of a preparation containing tafamidis may contain, for example 20 mg (12.2 mg as tafamidis).

The solid preparation containing tafamidis according to one embodiment of the present invention is provided as a powdered preparation and a tablet containing tafamidis, but is not limited thereto, and may include capsules, granules, powders and the like. The tablet containing tafamidis according to the present embodiment is not particularly limited as long as it is in the form of a tablet and includes orally disintegrating tablets, chewable tablets, effervescent tablets, dispersible tablets, and soluble tablets.

The powdered preparation containing tafamidis according to one embodiment of the present invention includes tafamidis, a dispersion medium, and a carrier.

The carrier according to one embodiment of the present invention includes, for example, one or more selected from a group consisting of magnesium aluminometasilicate, microcrystalline cellulose, low substituted hydroxypropyl cellulose, crospovidone and calcium silicate. Magnesium aluminometasilicate, microcrystalline cellulose, low substituted hydroxypropyl cellulose, crospovidone and calcium silicate are preferable because they have excellent elution rates of tafamidis from the preparation at pH 6.8. Magnesium aluminometasilicate, microcrystalline cellulose, low substituted hydroxypropyl cellulose and crospovidone have particularly excellent elution rates of tafamidis at pH 6.8 and are more preferred. Magnesium aluminometasilicate and calcium silicate are more preferable because they can adsorb the tafamidis dispersion in a small amount and have a high liquid holding capacity. Magnesium aluminometasilicate is particularly preferable because it can maintain the elution rate of tafamidis from the preparation at pH 6.8 and has a high liquid retention capacity. In the present embodiment, the specific carriers can form the solid preparations of tafamidis by adsorbing the tafamidis dispersion in which tafamidis is dispersed in the dispersion medium. Furthermore, the dissolution rate of tafamidis from the solid preparation can be maintained.

The dispersion medium for dispersing tafamidis according to one embodiment of the present invention includes, for example, polysorbate 80 and sorbitan monooleate. Polysorbate 80 and sorbitan monooleate are preferred as a dispersion medium. Further, the dispersion medium may contain water.

The powdered preparation containing tafamidis according to the present embodiment can be made into a solid preparation by dispersing tafamidis in the dispersion medium and adsorbing the tafamidis dispersion in the specific carriers, and the decrease in the elution rate of tafamidis from the preparation can be suppressed. The content of the carrier in the powdered preparation containing tafamidis is preferably 1% by mass or more and 5000% by mass or less with respect to tafamidis. The powdered preparation containing tafamidis preferably has a magnesium aluminometasilicate content of 1% by mass or more and 5000% by mass or less with respect to tafamidis.

In one embodiment, the powdered preparation containing tafamidis according to the present invention can be the tablet containing tafamidis together with the required additives.

Examples of the additive include excipients, binders, disintegrants, antioxidants, colorants, lubricants and the like. One kind of additive may be used alone, or two or more kinds may be used in the combination. Further, in the case of two or more kinds, a so-called premix additive, which is granulated by mixing a plurality of additives in advance, may be contained.

The excipients include, for example, starch, microcrystalline cellulose, cellulose derivatives and salts thereof, dextrin, lactose, mannitol, sorbitol, xylitol, trehalose, methacrylic acid copolymer, anhydrous dibasic calcium phosphate, sucrose, talc (Natural hydrous magnesium silicate), kaolin, precipitated calcium carbonate, sodium chloride, titanium oxide, light anhydrous silicic acid, magnesium aluminometasilicate and the like, but are not limited thereto.

The binders include, for example, starch, dextrin, tragant, gelatin, povidone, polyvinyl alcohol, hydroxypropyl cellulose, microcrystalline cellulose, hypromellose, ethyl cellulose, carmellose, methacrylic acid copolymer, sucrose, starch syrup, arabic rubber and the like, but are not limited thereto.

The disintegrants include, for example, starch, microcrystalline cellulose, carmellose calcium, crospovidone, low substituted hydroxypropyl cellulose, sodium starch glycolate, croscarmellose sodium, powdered agar and the like, but are not limited thereto.

The antioxidants include, for example, butylhydroxytoluene (BHT), propyl gallate, butylhydroxyanisole (BHA), recithin, α-tocopherol, hydroquinone, octyl gallate, dodecyl gallate, isoamyl gallate, nordihydroguaiaretic acid, gum guaicum, α-naphthylamine, ethyl protocatechuate (EPG), ascorbic acid, ascorbyl stearate, ascorbyl palmitate, cysteine hydrochloride, sodium ascorbic acid stearate, thioglycerol, thiosorbitol and the like, but are not limited thereto.

The colorants include, for example, iron sesquioxide, yellow ferric oxide, tar color and the like, but are not limited thereto.

The lubricant includes, for example, talc, starch, magnesium stearate, calcium stearate, sodium stearyl fumarate, boric acid, paraffin, cocoa butter, macrogol, leucine, sodium benzoate and the like, but are not limited thereto.

The tablet containing tafamidis according to the present embodiment can be produced by mixing the powdered preparation containing tafamidis and the additive, and tableting the obtained mixture to suppress a decrease in the elution rate of tafamidis from the preparation. The tablet containing tafamidis preferably has a content of the carrier of 1% by mass or more and 50% by mass or less per tablet. The tablet containing tafamidis preferably has a magnesium aluminometasilicate content of 1% by mass or more and 50% by mass or less per tablet.

In the method for producing the tablet containing tafamidis according to the present embodiment, the powdered preparation containing tafamidis is first produced, and then a mixture containing the powdered preparation containing tafamidis is tableted to produce the tablet containing tafamidis. The tablet containing tafamidis according to the present embodiment can be produced according to a production method known in the pharmaceutical field.

As a method for producing the powdered preparation containing tafamidis according to the present embodiment, first, tafamidis is dispersed in the dispersion medium. The tafamidis dispersion is then adsorbed in the carrier. At this time, another additive may be mixed with the carrier, and the tafamidis dispersion may be adsorbed in this mixture. The powdered preparation containing tafamidis may be further dried and sized.

As a method of adsorbing the tafamidis dispersion in the carrier, a method of adsorbing the tafamidis dispersion in the carrier by mixing, a method of adsorbing the tafamidis dispersion in the carrier by spraying, and a method of adsorbing the carrier in the tafamidis dispersion by mixing are available but are not limited thereto. By the above steps, the composition containing tafamidis according to the present invention can be produced.

The tablet containing tafamidis according to the present embodiment can be produced by mixing an additive with the powdered preparation containing tafamidis and tableting. The manufacturing method includes, but is not limited to, a kneading method, a fluidized bed granulation method, and a direct tableting method. The tablets formed by tableting may be further film-coated, for example.

### EXAMPLE

An example of a method for producing the solid preparation containing tafamidis according to the present invention, particularly a powdered preparation and a tablet, is shown below, but the description is merely an example, and the present invention is not limited thereto.

### (Producing the Powdered Preparation Containing Tafamidis)

20 mg of tafamidis meglumine was dispersed in a mixed liquid of 567 mg of macrogol 400, 75 mg of polysorbate 80, and 8 mg of sorbitan monooleate. 670 mg of the tafamidis dispersion in which tafamidis meglumine is dispersed in the mixed liquid and 700 mg or 3000 mg of each carrier were mixed in a mortar to obtain the powdered preparation containing tafamidis. Table 1 shows the types and mixing amounts of each carrier.

TABLE 1

| | Adsorption carrier (grade) | Mixing amount |
|---|---|---|
| Example 1 | Magnesium aluminometasilicate | 700 mg |
| | (UFL2) | |
| Example 2 | Microcrystalline cellulose | 700 mg |
| | (KG1000) | |
| Example 3 | Low substituted hydroxypropyl | 700 mg |
| | cellulose (NBD-022) | |
| Example 4 | Crospovidone (CL-M) | 700 mg |
| Example 5 | Calcium silicate (FLORITE) | 700 mg |
| Comparative | D-mannitol | 3000 mg |
| Example 1 | (mannit P) | |
| Comparative | Light anhydrous silicic acid | 700 mg |
| Example 2 | (AEROSIL 200) | |
| Comparative | Hydrated silicon dioxide | 700 mg |
| Example 3 | (Carplex # 80) | |

In Examples 1 to 5 and Comparative Examples 2 and 3, the tafamidis dispersion was sufficiently adsorbed and powdered. The powdered preparations containing tafamidis using magnesium aluminometasilicate and calcium silicate as carriers according to Examples 1 and 5 were able to adsorb the tafamidis dispersion with a small amount of carrier, and the liquid holding capacity of the carrier was particularly high. The oil absorption of magnesium aluminometasilicate is 2.7 to 3.4 mL/g, and the oil absorption of calcium silicate is 4.6 mL/g. On the other hand, the powdered preparation containing tafamidis using D-mannitol as a carrier according to Comparative Example 1 could not be powdered because the tafamidis dispersion could not be completely adsorbed when the amount of D-mannitol was 700 mg, which was the same as that of other carriers. When D-mannitol according to Comparative Example 1 was used as a carrier, 3000 mg of D-mannitol was added so that it could just be handled as a powder.

### (pH Shift Dissolution Method)

The tafamidis dispersion and the powdered preparation containing tafamidis according to Examples 1 to 5 and Comparative Examples 1 to 3 were stirred at 37° C. (paddle speed 150 rpm/min) in 500 ml of the 1^{st} fluid (pH 1.2) for dissolution test and sampling was performed over time (0, 5, 15, 30 minutes) for 30 minutes. The obtained samples were filtered through a membrane filter having a pore size of 0.45 μm, the residue was dissolved in methanol, and the absorbance at a wavelength of 310 nm was measured by a UV measurement method (referred to as a sample of pH 1.2).

Then, the pH was changed to 6.8 by adding sodium hydroxide and the aqueous solution of potassium dihydrogen phosphate, and sampling was performed over time (45, 60, 90, 120 minutes) with stirring (paddle speed 50 rpm/min) at 37° C. for 120 minutes. The obtained samples were filtered through a membrane filter having a pore size of 0.45 μm, the residue was dissolved in methanol, and the absorbance at a wavelength of 310 nm was measured by a UV measurement method (referred to as a sample of pH 6.8). The elution rate from the Tafamidis preparation at each time was calculated with the elution rate after 0 minutes as 0%.

Table 2 shows the dissolution rate (%) from the tafamidis preparation of the tafamidis dispersion and the powdered preparations containing tafamidis according to Examples 1 to 5 and Comparative Examples 1 to 3.

TABLE 2

| | pH 1.2 | | | | pH 6.8 | | | |
|---|---|---|---|---|---|---|---|---|
| Time (min) | 0 | 5 | 15 | 30 | 45 | 60 | 90 | 120 |
| Example 1 | 0 | 0 | 0.3 | 0.2 | 91.5 | 94.1 | 94.1 | 95.5 |
| Magnesium | | | | | | | | |
| aluminometasilicate | | | | | | | | |
| Example 2 | 0 | 7.4 | 3.8 | 2.6 | 91 | 94.7 | 97.1 | 99.1 |
| Microcrystalline | | | | | | | | |
| cellulose | | | | | | | | |
| Example 3 | 0 | 8.1 | 4.4 | 2.8 | 98.8 | 99.7 | 99.8 | 100.2 |
| Low substituted | | | | | | | | |
| hydroxypropyl | | | | | | | | |
| cellulose | | | | | | | | |
| Example 4 | 0 | 6.1 | 3.0 | 2.2 | 97.1 | 98.6 | 99.9 | 100.2 |
| Crospovidone | | | | | | | | |
| Example 5 | 0 | 0.3 | 0.3 | 0.2 | 77.5 | 82.6 | 88.9 | 91.3 |
| Calcium | | | | | | | | |
| silicate | | | | | | | | |
| Comparative | 0 | 5.7 | 2.5 | 2.1 | 97.2 | 98.1 | 98 | 98.3 |
| Example 1 | | | | | | | | |
| D-mannitol | | | | | | | | |
| Comparative | 0 | 0.3 | 0.3 | 0.2 | 63.7 | 69.6 | 73.6 | 75.6 |
| Example 2 | | | | | | | | |
| light | | | | | | | | |
| anhydrous | | | | | | | | |
| silicic acid | | | | | | | | |
| Comparative | 0 | 0.4 | 0.3 | 0.2 | 67.5 | 71.8 | 75.4 | 76.4 |
| Example 3 | | | | | | | | |
| Hydrated | | | | | | | | |
| silicon dioxide | | | | | | | | |
| Tafamidis | 0 | 6.6 | 3.5 | 2.6 | 98.2 | 100.7 | 100.7 | 101.6 |
| dispersion | | | | | | | | |

From the results in Table 2, the powdered preparation containing tafamidis using magnesium aluminometasilicate, microcrystalline cellulose, low substituted hydroxypropyl cellulose, crospovidone, calcium silicate or D-mannitol as a carrier according to Examples 1 to 5 and Comparative Example 1 were able to maintain an elution rate at pH 6.8, similar to the tafamidis dispersion. The powdered preparation containing tafamidis using light anhydrous silicic acid or hydrated silicon dioxide as a carrier according to Comparative Examples 2 and 3 had a lower elution rate at pH 6.8 as compared with the tafamidis dispersion. The powdered preparation containing tafamidis using magnesium aluminometasilicate as a carrier according to Example 1 was able to maintain the elution rate at pH 6.8 as in the tafamidis dispersion.

### (Producing the Tablet Containing Tafamidis)

The amounts of tafamidis and each additive in the production methods of Examples 6 to 8 are described as an amount per tablet.

### Example 6

20 mg of tafamidis meglumine was dispersed in a mixed liquid of 75 mg of polysorbate 80 and 8 mg of sorbitan monooleate. 103 mg of tafamidis dispersion in which tafamidis meglumine was dispersed in the mixed liquid and 103 mg of magnesium aluminometasilicate were mixed in a mortar to obtain the powdered preparation containing tafamidis. 216 mg of anhydrous dibasic calcium phosphate, 250 mg of microcrystalline cellulose, 21 mg of croscarmellose sodium, and 7 mg of magnesium stearate were added to the powdered preparation containing tafamidis, mixed, and tableted at 700 mg per tablet.

### Example 7

20 mg of tafamidis meglumine was dispersed in a mixed liquid of 75 mg of polysorbate 80, 8 mg of sorbitan monooleate, and 80 mg of purified water. 183 mg of tafamidis dispersion in which tafamidis meglumine was dispersed in the mixed liquid and 103 mg of magnesium aluminometasilicate were mixed in a mortar. The resulting mixture was dried in a shelf dryer. 216 mg of anhydrous dibasic calcium phosphate, 250 mg of microcrystalline cellulose, 21 mg of croscarmellose sodium, and 7 mg of magnesium stearate were added to the dried powdered preparation containing tafamidis, mixed, and tableted at 700 mg per tablet.

### Example 8

20 mg of tafamidis meglumine was dispersed in the mixed liquid of 75 mg of polysorbate 80, 8 mg of sorbitan monooleate, and 200 mg of purified water. 103 mg of magnesium aluminometasilicate, 100 mg of anhydrous dibasic calcium phosphate and 100 mg of microcrystalline cellulose were added to a fluidized bed granulator dryer, and 303 mg of tafamidis meglumine dispersion was sprayed to the additive containing carrier and dried. 116 mg of anhydrous dibasic calcium phosphate, 150 mg of microcrystalline cellulose, 21 mg of croscarmellose sodium, and 7 mg of magnesium stearate were added to the obtained powdered preparation containing tafamidis, mixed, and tableted at 700 mg per tablet.

Table 3 shows the compositions of the tablet containing tafamidis according to Examples 6 to 8.

TABLE 3

| | | Example 6 | Example 7 | Example 8 |
|---|---|---|---|---|
| | | Direct | Kneading | Fluidized |
| | Grade | tableting (mg) | (mg) | bed (mg) |
| Tafamidis | — | 20.0 | 20.0 | 20.0 |
| meglumine | | | | |
| Sorbitan | SV-10 | 8.0 | 8.0 | 8.0 |
| monooleate | | | | |
| Polysorbate 80 | — | 75.0 | 75.0 | 75.0 |
| Purified water | — | — | (80) | (200) |
| API dispersion | — | 103.0 | 103.0 | 103.0 |
| subtotal | | | (183.0) | (303.0) |
| Magnesium | Neusilin | 103.0 | 103.0 | 103.0 |
| aluminometasilicate | UFL2 | | | |
| Anhydrous dibasic | GS Calica | — | — | 100.0 |
| calcium phosphate | | | | |
| Microcrystalline | UF702 | — | — | 100.0 |
| cellulose | | | | |
| Adsorption grain | — | 206.0 | 206.0 | 406.0 |
| subtotal | | | | |
| Anhydrous dibasic | GS Calica | 216.0 | 216.0 | 116.0 |
| calcium phosphate | | | | |
| Microcrystalline | UF702 | 250.0 | 250.0 | 150.0 |
| cellulose | | | | |
| Croscarmellose | Ac-di-sol | 21.0 | 21.0 | 21.0 |
| sodium | | | | |
| Magnesium | — | 7.0 | 7.0 | 7.0 |
| stearate | | | | |
| Total | — | 700.0 | 700.0 | 700.0 |

### (pH Shift Dissolution Method)

The tablet containing tafamidis according to Examples 6 to 8 were subjected to a dissolution test by pH shift dissolution method in the same manner as the above-mentioned powdered preparations containing tafamidis. Table 4 shows the dissolution rates of the tafamidis from the tablet containing tafamidis according to Examples 6 to 8.

TABLE 4

| | pH 1.2 | | | | pH 6.8 | | | |
|---|---|---|---|---|---|---|---|---|
| Time (min) | 0 | 5 | 15 | 30 | 45 | 60 | 90 | 120 |
| Example 6 | 0 | 3.9 | 2.8 | 1.7 | 79.2 | 84.4 | 89.2 | 92.1 |
| (Direct | | | | | | | | |
| tableting | | | | | | | | |
| method) | | | | | | | | |
| Example 7 | 0 | 3.4 | 2.9 | 1.9 | 84.8 | 89.1 | 94.2 | 95.7 |
| (Kneading | | | | | | | | |
| method) | | | | | | | | |
| Example 8 | 0 | 3.7 | 2.8 | 1.8 | 90.3 | 94.0 | 96.7 | 97.6 |
| (Fluidized bed | | | | | | | | |
| granulation | | | | | | | | |
| method) | | | | | | | | |

From the results in Table 4, all of the tablet containing tafamidis produced by the direct tableting method, the kneading method or the fluidized bed granulation method according to Examples 6 to 8 were able to improve the elution rate at pH 6.8.

Even if other working effects which are different from the working effect brought about by the aspect of each above-mentioned embodiment, if it is clear from the description in this specification, or can be easily predicted by the person skilled in the art, then it is naturally understood to be brought about by the present invention.

What is claimed is:

1. A solid preparation containing tafamidis comprising:
tafamidis; and
a carrier containing one or more selected from a group consisting of magnesium aluminometasilicate, microcrystalline cellulose, low substituted hydroxypropyl cellulose, crospovidone and calcium silicate.

2. The solid preparation containing tafamidis according to claim 1 further comprising polysorbate 80 and sorbitan monooleate.

3. The solid preparation containing tafamidis according to claim 1, wherein
the carrier contains one or more selected from a group consisting of magnesium aluminometasilicate, microcrystalline cellulose, low-substituted hydroxypropyl cellulose and crospovidone.

4. The solid preparation containing tafamidis according to claim 2, wherein
the carrier contains one or more selected from a group consisting of magnesium aluminometasilicate, microcrystalline cellulose, low-substituted hydroxypropyl cellulose and crospovidone.

5. The solid preparation containing tafamidis according to claim 1, wherein
the carrier is magnesium aluminometasilicate.

6. The solid preparation containing tafamidis according to claim 2, wherein
the carrier is magnesium aluminometasilicate.

7. The solid preparation containing tafamidis according to claim 1, wherein
the solid preparation containing tafamidis is a powdered preparation or a tablet.

8. The solid preparation containing tafamidis according to claim 2, wherein
the solid preparation containing tafamidis is a powdered preparation or a tablet.

9. The solid preparation containing tafamidis according to claim 3, wherein
the solid preparation containing tafamidis is a powdered preparation or a tablet.

10. The solid preparation containing tafamidis according to claim 4, wherein
the solid preparation containing tafamidis is a powdered preparation or a tablet.

11. The solid preparation containing tafamidis according to claim 5, wherein
the solid preparation containing tafamidis is a powdered preparation or a tablet.

12. The solid preparation containing tafamidis according to claim 6, wherein
the solid preparation containing tafamidis is a powdered preparation or a tablet.

13. A method for producing a solid preparation containing tafamidis, comprising:
dispersing tafamidis in a dispersion medium to obtain a tafamidis dispersion, and
adsorbing the tafamidis dispersion in a carrier containing one or more selected from a group consisting of magnesium aluminometasilicate, microcrystalline cellulose, low substituted hydroxypropyl cellulose, crospovidone and calcium silicate.

14. The method for producing a solid preparation containing tafamidis according to claim 13, wherein
the dispersion medium includes polysorbate 80 and sorbitan monooleate.

15. The method for producing a solid preparation containing tafamidis according to claim 13, wherein
the carrier contains one or more selected from a group consisting of magnesium aluminometasilicate, microcrystalline cellulose, low-substituted hydroxypropyl cellulose and crospovidone.

16. The method for producing a solid preparation containing tafamidis according to claim 14, wherein
the carrier contains one or more selected from a group consisting of magnesium aluminometasilicate, microcrystalline cellulose, low-substituted hydroxypropyl cellulose and crospovidone.

17. The method for producing a solid preparation containing tafamidis according to claim 13, wherein
the carrier is magnesium aluminometasilicate.

18. The method for producing a solid preparation containing tafamidis according to claim 14, wherein
the carrier is magnesium aluminometasilicate.

19. A method for producing a tablet containing tafamidis, comprising:
mixing a powder preparation containing tafamidis and an additive to obtain a mixture and tableting the mixture, wherein
the powder preparation containing tafamidis is the solid preparation containing tafamidis produced by the method for producing the solid preparation containing tafamidis according to claim 13.

20. A method for producing a tablet containing tafamidis, comprising:
mixing a powder preparation containing tafamidis and an additive to obtain a mixture and tableting the mixture, wherein
the powder preparation containing tafamidis is the solid preparation containing tafamidis produced by the method for producing the solid preparation containing tafamidis according to claim 14.
